(19) United States
(12) Patent Application Publication
(10) Pub. No.: US 2014/0081356 A1
(43) Pub. Date: Mar. 20, 2014

(54) THERMAL CAPACITORS FOR MINIMIZING COMPLICATIONS AND SIDE EFFECTS FROM THERMAL MEDICINE

(71) Applicant: GEORGIA TECH RESEARCH CORPORATION, Atlanta, GA (US)

(72) Inventors: Andrei G. Fedorov, Atlanta, GA (US); Craig Elkton Green, Atlanta, GA (US)

(73) Assignee: GEORGIA TECH RESEARCH CORPORATION, Atlanta, GA (US)

(21) Appl. No.: 13/833,455

(22) Filed: Mar. 15, 2013

Related U.S. Application Data
(60) Provisional application No. 61/703,545, filed on Sep. 20, 2012.

(51) Int. Cl. A61N 5/06

(52) U.S. Cl. CPC A61N 5/062
USPC 607/88; 607/96

(57) ABSTRACT

A system for controlling temperatures during medical procedures is disclosed. The system can include a high-thermal conductivity matrix with embedded thermal capacitors. The matrix can be a cream or gel, for example, and can enable the system to be easily applied to the skin, or other area to be treated. The thermal capacitors can include phase change materials, polymers, fats, or other materials that undergo an endothermic physico-chemical transformation above a first predetermined temperature (e.g., room temperature), but below a second predetermined temperature (e.g., the patient's pain threshold). The system can further include one or more thickeners to provide the desired rheological properties for application. The system can also include one or more lubricants to ease some operations during the medical procedure.

### CROSS REFERENCE TO RELATED APPLICATIONS

This application claims priority to and benefit under 35 USC §119(e) of U.S. Provisional Patent Application Ser. No. 61/703,545, of the same title, filed Sep. 20, 2012, which is hereby incorporated by reference as if fully set forth below.

### BACKGROUND OF THE INVENTION

1. Field of the Invention

Embodiments of the present invention relates generally to heat absorbing compounds, and more specifically to heat absorbing compounds comprising one or more thermal capacitors disposed in a high heat capacity matrix for thermal control during medical procedures.

2. Background of Related Art

Non ablative laser and pulsed light medicine is an important tool for treating a number of skin diseases including, but not limited to, acne, eczema, vascular lesions, and scar removal. Photodynamic therapy, a light based photo destructive therapy, for example, is a nonsurgical alternative used to treat cancerous and pre-cancerous tumors. Photodynamic therapy has also been shown to be effective in treating severe acne.

While effective, these laser and light based treatments can cause significant to severe pain and discomfort to the patient during the procedure due to the buildup of heat on the skin caused by the energy source. In some instances, thermal injury and burns may occur as side effects of dermatological treatments, for example, resulting in scarring, edema, and discoloration, among other thing. While these injuries are sometimes temporary, they can also be permanent, and are preventable with adequate cooling.

Topical anesthetics, such as EMLA® cream, for example, can be effective for pain control, but do little to regulate temperature. In addition, they have the potential for adverse side effects such as allergic reactions, redness, and swelling. In addition, these products typically take an hour or more to become effective, which can adversely affect clinic scheduling and throughput, particularly when sensitive patients require anesthetic unexpectedly.

For thermal control, ultrasound gel is sometimes applied to the skin prior to treatment. Unfortunately, while ultrasound gel can absorb some thermal energy, it is not designed for this purpose. As a result, ultrasound gel is moderately effective at best. As a result, many patients using only ultrasound gel still experience significant pain and discomfort during treatment.

Other methods of supplying supplementary cooling, including air or cryogenic spray cooling, have shown to be somewhat effective. These systems are also bulky and expensive; however, and can reduce treatment effectiveness due to overcooling. A hydrogel based patch containing approximately 95% water, on the other hand, shows some effectiveness in reducing thermally induced pain during laser therapy.¹ The morphology of a patch, as opposed to a spreadable fluidic gel, limits the scalability of this type of solution, however, due to the broad variation in treatment site sizes and shapes. Furthermore, conventional solutions are only capable of energy storage through sensible heating (i.e., a change in temperature of the solution), which significantly limits the heat capacity per unit volume of thermal solution. Energy storage through phase change or other endothermic physical and/or chemical (physico-chemical) change, on the other hand, provides much higher heat absorption capacity with little or no change in temperature. ¹D. Cassuto, J. F. Mollia, L. Scrimali, and P. Siragò, “Right-left comparison study of hydrogel pad versus transparent fluid gel in patients with dermo-cosmetic lesions undergoing non-ablative laser therapy,” Journal of Cosmetic and Laser Therapy, vol. 11, pp. 45-51, 2009.

What is needed, therefore, is a system for providing enhanced thermal control for various medical procedures to minimize pain and thermal damage experienced during laser and light based medical procedures, for example. The system should be liquid or gel bases to enable a broad range of applications and application sites. The system can include a gel or liquid matrix with high inherent heat capacity (i.e., a sensible component) and imbedded thermal capacitors to enable heat absorption through material phase change. It is to such a system that embodiments of the present invention are primarily directed.

### BRIEF SUMMARY OF THE INVENTION

Embodiments of the present invention relate generally a composite gel, liquid, or cream comprising a high thermal conductivity matrix carrier with embedded thermal capacitors comprising a material that undergoes an endothermic physico-chemical transformation at or above a predetermined temperature to provide heat absorption through a change of the physical or chemical state of one or more components (e.g., phase change). In some embodiments, the carrier can comprise a gel, liquid, or cream to enable the system to be applied topically or injected. The carrier can comprise a material with a high heat capacity material. The carrier can be supplemented with a plurality of thermal capacitors. The thermal capacitors can undergo one or more endothermic physico-chemical transformations to absorb heat during medical treatments (e.g., lasers) of the body parts accompanied by heat generation/release.

In some embodiments, the thermal capacitors can comprise phase change materials. In other embodiments, the thermal capacitors can comprise materials that decompose or denature. In still other embodiments, the thermal capacitors can comprise multiple components that undergo an endothermic chemical reaction. Regardless, the thermal capacitors can absorb heat by virtue of an endothermic physico-chemical transformation to control the temperature of a thermal procedure at a substantially constant rate.

Embodiments of the present invention can comprise a system including a conformable, high thermal conductivity matrix, with high thermal conductivity and high sensible heat capacity, and a plurality of thermal capacitors with high latent heat capacity disposed in the matrix such that there is high contact area between the thermal capacitors and the matrix, which can control the temperature of a portion of the patient's body during a medical procedure. In some embodiments, the thermal capacitors can be phase change materials (PCMs) and can undergo an endothermic physico-chemical change from a first state to a second state.

In some embodiments, the PCMs can be a solid suspended in the conformable matrix below the first predetermined temperature and can undergo a phase change from solid to liquid above the first predetermined temperature. In other embodiments, the PCMs can be a liquid and can be dissolved, suspended, or both in the conformable matrix below the first predetermined temperature. In this configuration, the endothermic physico-chemical change can be a phase change from liquid to gas above the first predetermined temperature.

In some embodiments, the medical procedure can be a light-based medical procedure in at a first predetermined wavelength range. In this configuration, the system can be substantially transparent in the first predetermined wavelength range. The medical procedure can be an intense pulsed light (IPL) treatment, for example, and the first predetermined wavelength range can be between approximately 500 nm and 1200 nm. In other embodiments, the medical procedure is a laser treatment at a first wavelength, due to laser lights substantially coherent light. In this configuration, the first predetermined wavelength range can be equivalent to the wavelength of the laser.

In some embodiments, the components of the system can be hypoallergenic, non-toxic, or both. In other embodiments, the PCMs can be, for example and not limitation, fatty acids, fatty acid esters, salt hydrates, and waxes. In some embodiments, the system can further comprise one or more thickeners to adjust the rheological properties of the system or one or more high thermal conductivity additives to enhance the thermal conductivity of the system.

Embodiments of the present invention can also comprise a system including a conformable, high thermal conductivity patch, with high thermal conductivity and high sensible heat capacity, and a plurality of thermal capacitors with high latent heat capacity disposed in the conformable patch such that there is high contact area between the thermal capacitors and the patch. In some embodiments, the patch can be appliable to a surface of a patient's body. As before, the thermal capacitors can undergo an endothermic physico-chemical reaction above a first predetermined temperature and below a second predetermined temperature to control the temperature of a portion of the patient's body during a medical procedure. In some embodiments, the patch can further define a hole disposed approximately in the center of the patch.

In some embodiments, the first predetermined temperature can be room temperature, or approximately 20° C. In other embodiments, the second predetermined temperature can be between approximately 44-60° C., the temperature associated with patient pain and/or injury to under normal circumstances.

Embodiments of the present invention can also comprise a method including the steps of applying a gel to a portion of the patient's skin and irradiating the patient's skin with a light-based therapy. The gel can comprise a conformable, high thermal conductivity matrix, with high thermal conductivity and high sensible heat capacity, and a plurality of thermal capacitors with high latent heat capacity disposed in the matrix such that there is high contact area between the thermal capacitors and the matrix. As discussed above, the thermal capacitors can undergo an endothermic physico-chemical reaction above a first predetermined temperature and below a second predetermined temperature and can control the temperature of the portion of the patient's skin during the light-based therapy.

During some procedures, the gel can only be applied to the portion of the patient's skin that is not being treated with the light-based therapy. In other embodiments, the light-based therapy can comprise light in a first pre-determined wavelength range and the gel can be substantially transparent in the first pre-determined wavelength range. In other examples, applying the gel to a portion of the patient's skin can comprise injecting the gel sub-epidermally with a plurality of microneedles.

These and other objects, features and advantages of the present invention will become more apparent upon reading the following specification in conjunction with the accompanying drawing figures.

### BRIEF DESCRIPTION OF THE DRAWINGS

FIG. 1a depicts a topical application of the system, in accordance with some embodiments of the present invention.

FIG. 1b depicts a detailed view of the system from FIG. 1a, in accordance with some embodiments of the present invention.

FIG. 2 is a graph depicting the effect the system has on skin temperature, in accordance with some embodiments of the present invention.

FIG. 3 is a graph depicting experimental results of the effect the system has on skin temperature using a pulsed heat source, in accordance with some embodiments of the present invention.

FIG. 4a depicts a topical application of the system with a transparent patch, in accordance with some embodiments of the present invention.

FIG. 4b depicts a topical application of the system with a toroidal patch, in accordance with some embodiments of the present invention.

FIG. 5 depicts a subcutaneous application of the system, in accordance with some embodiments of the present invention.

FIG. 6 depicts another topical application of the system with a viscous gel, in accordance with some embodiments of the present invention.

### DETAILED DESCRIPTION OF THE INVENTION

Embodiments of the present invention relates generally to heat absorbing compounds, and more specifically to heat absorbing compounds comprising one or more thermal capacitors disposed in a high heat capacity matrix for temperature control during medical procedures. The system disclosed herein can be a topically applied thermal storage medium that can significantly reduce the pain associated with medical procedures including, for example, non-ablative laser and light based therapies. The system can include a conformable, high heat-capacity matrix (e.g., a gel) with embedded thermal capacitors comprising phase change materials (PCMs) or other endothermic materials.

To simplify and clarify explanation, the system is described below as a “gel.” One skilled in the art will recognize, however, that the invention is not so limited. The system can also comprise a patch, cream, liquid, suspension, emulsion, hydrogel, or other form of topical or subcutaneous application. Thus “gel” is understood as shorthand for the full range of potential physical embodiments of this invention. Furthermore, while generally referred to below as a gel for topical application, other skin applications including application to the epidermis, dermis, and subcutis and other non-skin related applications are contemplated herein. Delivery to sub-epidermal layers of the skin may occur through, for example, microneedle injection, traditional injection, absorption, skin permeation, or other techniques.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

A number of medical and dermatological procedures (e.g., laser or photodynamic therapy) produce excess heat on the skin. Heat in excess of a particular patient's pain threshold obviously causes the patient discomfort. In addition, the excess heat build-up can cause temporary or permanent injuries including, but not limited to, redness, swelling, and scarring. A problem with conventional topical applications for these procedures has been that they are not design to absorb significant amounts of heat. As mentioned above, ultrasound gel has been used, for example, but provides only sensible heat absorption, which severely limits its heat absorption capabilities and also results in continuous temperature increase. Other conventional solutions, including hydrogel based patches, for example, provide similarly limited capabilities.

When materials change phase (e.g., melt), however, transitioning from one state to another (e.g., from solid to liquid), they absorb a great deal of energy without an associated change in temperature. This phenomenon exploits the material property referred to as a material's latent heat of phase transformation, which is well known for numerous materials. Materials with particularly high latent heats can be powerful tools for controlling the temperature of surfaces that are heated in a transient or pulsed nature. The transient nature of the heating can be important because once all of the material has melted, the desired effect is lost.

In dermatology, for example, a pulsed or transient light source such as a laser or intense pulsed light (IPL) device can be used to remove blemishes or other unwanted facial features, or to change the skin's texture or tightness. Along with these desirable effects, the laser or light can also rapidly heat the skin causing the patient a significant amount of pain and discomfort. Because the excess thermal energy in non-ablative laser procedures, for example, does not serve a primary medical function, its removal from the skin would significantly reduce the pain associated with the treatment without negatively affecting the positive effects.

Embodiments of the present invention, therefore, can comprise a plurality of solid to liquid PCMs in a clear, or semi-transparent, gel matrix. When the PCM loaded gel is applied to the skin, it absorbs the excess heat created by the laser and uses that excess heat to melt the PCMs, preventing what would typically be a rapid temperature rise. PCM based composites have been used in existing technologies such as, for example, embedded components of electronic devices and are capable of increasing by up to a factor of 20× the amount of time that a pulsed device such as a laser can operate at load before exceeding internal temperature thresholds.² Alternately, the same device (e.g., laser) can increase its power delivery by a factor of 10 or more in a given time period without exceeding allowable skin temperatures. ²C. E. Green, A. G. Fedorov, and Y. K. Joshi, “Thermal capacitance matching in 3D many-core architectures,” in 27th Annual IEEE SEMI-THERM Symposium, San Jose, 2011, pp. 110-115; U.S. Pat. No. 8,378,453.

In some embodiments, the system 100 can comprise biocompatible and/or minimally irritating materials. In other embodiments, such as for particularly harsh or irritating procedures or patients with sensitive skin, hypoallergenic materials can be used. In configurations using topical applications to skin, for example, it can be advantageous for the system to comprise minimally irritating components to prevent skin reactions and other side effects. In configurations using subcutaneous or other internal applications, all materials are preferably biocompatible such that they can be absorbed, dissolved, excreted, or otherwise safely removed by the body with minimal, or no, side effects.

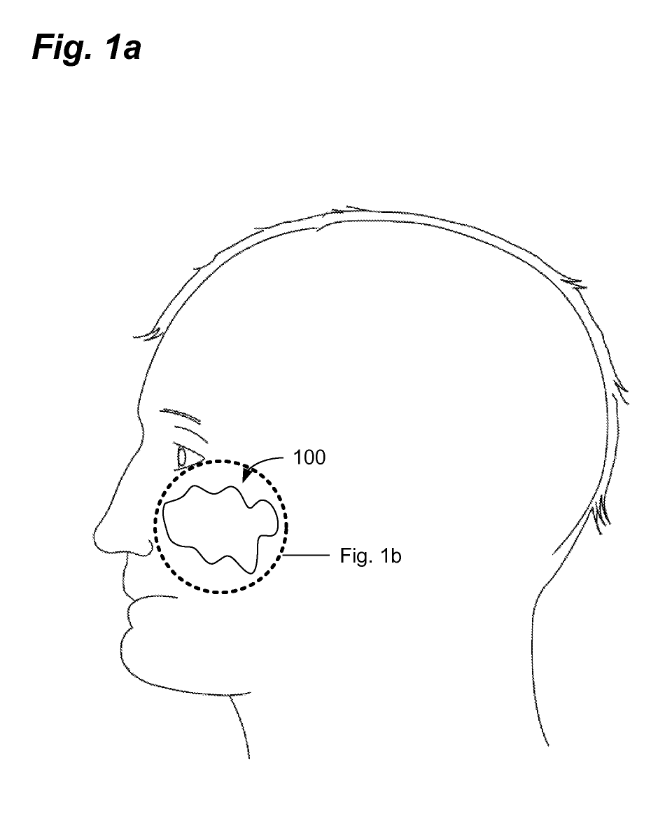

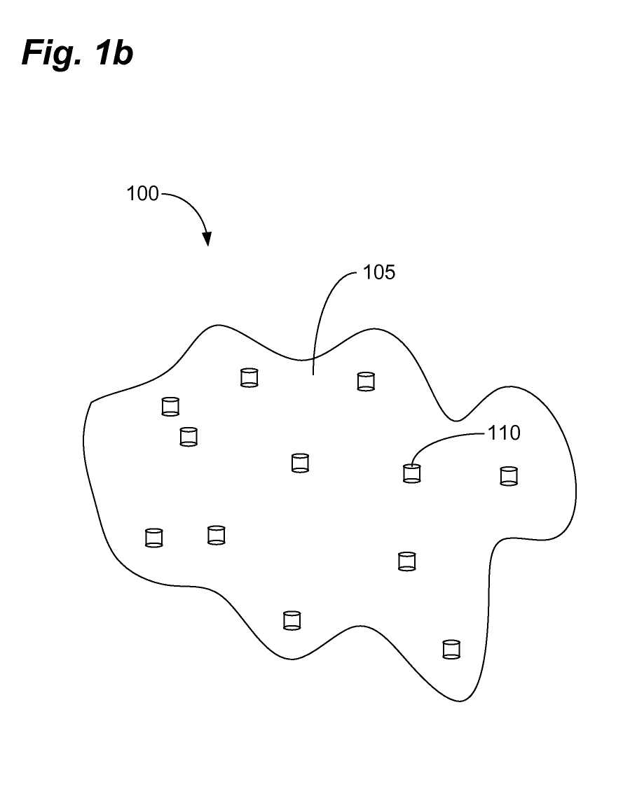

To this end, as shown in FIGS. 1a and 1b, embodiments of the present invention relate to a system 100 for absorbing heat and controlling temperatures during various medical procedures. In some embodiments, the system can comprise a composite material 100 comprising a conformable material 105 such as, for example and not limitation, a gel, liquid, or cream with embedded thermal capacitors 110. The gel 105 portion of the system can provide, for example, adhesion to the body, lubricity for surgical tools, and high heat transfer, among other things. The thermal capacitors 105, on the other hand, can comprise a phase change material (PCM), for example, or other materials that undergo endothermic physico-chemical transformations at the desired temperatures.

In this manner, the system 100 can provide improved heat absorption and temperature control by absorbing heat using two mechanisms. The first mechanism is sensible heating, or the heat required to raise the temperature of the system 100. The second mechanism is the heat required to change the phase of the thermal capacitors 110, or latent heat. The second mechanism is particularly effective because the material for the thermal capacitors 110 can be chosen to have a high latent heat (i.e., materials for which it requires large amounts of energy to affect the change). This is particularly effective because during phase transition, no change in temperature occurs. In this manner, the material for the capacitors 110 can be designed such that their melting or boiling point is above room temperature, for example, but below the temperature at which pain and/or injury occurs for the patient.

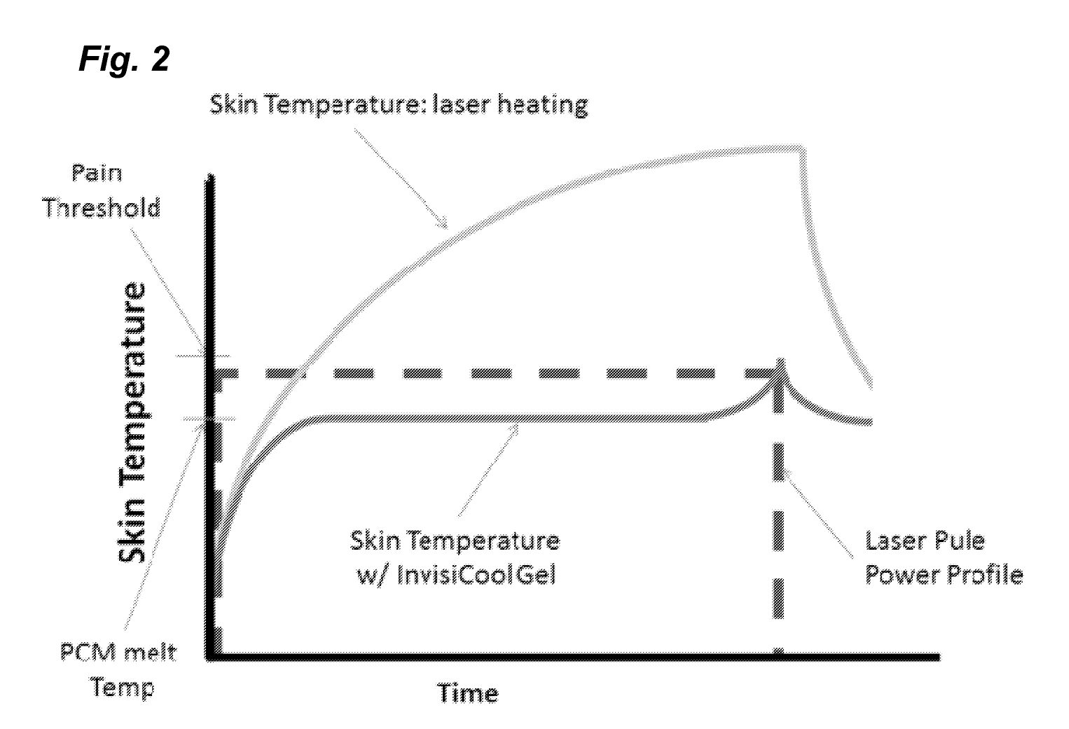

As shown in FIG. 2, using conventional means, immediately after the laser pulse is initiated, the temperature of the patient's skin begins to rise, quickly reaching and then exceeding the pain threshold, which is generally in the range of approximately 44-60° C. depending on the duration of exposure.³ Embodiments of the present invention, however, provide improved thermal control. With the system 100 applied and the patient subjected to the same laser pulse, the patient's skin temperature begins to rise just as in the previous case. Once the skin reaches the PCM 110 melting temperature, however, the temperature rise stops and that energy is instead used to melt the PCMs 110. The skin remains at an approximately constant temperature until the PCMs 110 are exhausted (e.g., fully melted), when temperature again begins to rise. ³Pertovaara, Antti, Timo Kauppila, and Minna M. Hämäläinen, “Influence of skin temperature on heat pain threshold in humans,” Experimental brain research 107.3 (1996): 497-503.

Advantageously, the system 100 can enable the skin to remain below the patient's pain threshold and/or the threshold for temporary or permanent injury for the duration of the laser or light driven procedure. Cell death, for example, generally begins to occur when the epidermal layer reaches approximately 44° C. This reduction in temperature, in turn, can significantly reduce pain, swelling, and scarring caused by the procedure. In addition, the temperature and pain reduction is achieved without the need for significant skin sub-cooling (e.g., spray cooling systems), improving the laser's effectiveness, for example.

In some embodiments, the PCMs 110 can comprise, for example and not limitation, fatty acids, derivatives such as fatty acid esters, low melting temperature waxes or wax mixtures, hydrated salts or ionic liquids, alcohols, or glycols. In some embodiments, the PCM 110 can be a liquid at room temperature, for example, that is dissolved or suspended in the carrier matrix of the gel 105. In this configuration, the PCM 110 can undergo, for example, a transition from liquid to vapor during the medical procedure, while absorbing excess thermal energy from the treatment site.

In other embodiments, the PCMs 110 can comprise materials that do not undergo a typical state change (e.g., solid to liquid or liquid to vapor), but rather undergo change in structure or decomposition. These changes can include, for example, a protein or blood plasma which undergoes denaturing. In other examples, the PCMs 110 can comprise a polymer that changes from a crystalline or semi-crystalline state to an amorphous phase or decomposes into one or more monomers. In still other embodiments, the PCMs 110 can undergo a chemical reaction, for example, where the accompanying chemical reaction is endothermic, thus absorbing the excess thermal energy from the laser to support the reaction.

For use with light-based procedures (e.g., using lasers), the gel 100 can be substantially transparent with respect to the laser or light based irradiation to minimize interference with the functionality of the medical treatment. To this end, in some embodiments, the constituents of the gel can be primarily composed of non-absorbing or dielectric materials. In this configuration, light absorbing additives, such as metals and other components, can be kept in relatively small concentrations. In other embodiments, the significance of the concentration of absorbing materials can be minimized by providing sufficient thermal capacitance in the system 100 to enable very thin applications. In other words, if the thermal capacitance is sufficiently large to enable application of the gel in films that are thinner than the extinction length of the light source, the transparency of the system 100 is of minimal importance.

In some embodiments, the carrier 105 can have high heat capacity and a structural component to ensure that the gel or patch stays in place when applied. In some embodiments, the gel 105 can also be designed such that it does not insulate the patient's skin from the ambient air. To this end, water may serve as both the external medium 105 that supports the suspension or emulsion of PCM particles 110 and the means of enhancing the intrinsic thermal conductivity of the PCM 110. In other embodiments, other liquids such as alcohols or oils may also serve as the external medium 105 supporting the suspension or dissolution of PCMs 110 and other additives, especially when the thermal conductivity of the overall medium is enhanced through other additives.

In some embodiments, the thermal conductivity of the PCMs 110, and the system 100 as a whole, may be enhanced through the addition of high thermal conductivity particles in the carrier 105. These particles can include materials such as, for example and not limitation, thermally conducting polymers, metallic nano or micro particles, carbon based materials, or other high thermal conductivity materials.

In some embodiments, the structural integrity of the gel or patch 105 can be provided by additives that increase viscosity and/or stability. These can be natural additives such as, for example and not limitation, polysaccharides, their derivatives (e.g., xanthan or guar gum, cellulose, agar, etc.), or gelatin. In other examples, these can be synthetic thickeners including, but not limited to, polyvinyl alcohol, sodium polyacrylate, acrylate polymers, or other cross linking polymeric materials. These materials can be used in the formulation of hydrogels, creams, and thickeners, such as those used in baking and other cooking processes.

In some embodiments, because the system 100 is a multi-component mixture, an emulsifier or stabilizer, such a surfactant or detergent, can be used. These components can beneficially reduce the interfacial tension between the additives in the system 100 and the external medium 105 (e.g. water). In some formulations, the emulsifier may also provide repulsion, which can reduce the tendency for additives to agglomerate or can simply increase the viscosity of the system 100, among other things.

In some embodiments, the emulsifier may include materials such as cellulose esters like methylcellulose or hydroxypropyl methylcellulose polymers, glycerin fatty acid esters, esters of monoglycerides, various esters of fatty acids (e.g. sucrose esters or propylene glycol esters), polyglycerol polyricinoleate, calcium stearoyl di laciate, letchin and its derivatives, and other anionic, cationic, amphoteric, and non-ionic emulsifiers. Regardless of the emulsifier, many emulsifiers advantageously increase both viscosity (i.e., mechanical stability) and additive suspension.

In some embodiments, the viscosity of the gel can be controlled to ensure that it can be spread in a thick enough film to be an effective heat sink, yet not become bulky and intrusive when performing the procedure. In this vein, viscosity can be controlled through the use of thickeners and through the use of commercial lubricants, which can have thickening properties. Obviously, lubricants can also serve the secondary purpose of providing the gel with good lubricity. This can enable the medical device performing the procedure to glide easily across the surface of the skin, for example, ensuring that the medical instrument (e.g., laser) can be easily manipulated. In some embodiments, suitable lubricants can include, for example and not limitation, glycerol, petroleum and its derivatives, water, vegetable oils, esters, hydrogenated polyolefins, silicones, and fluorocarbons.

In some embodiments the gel can contain a biological control agent to reduce the growth of bacteria, algae, fungi or other living contaminants during storage. Suitable biological control agents can include, for example and not limitation, alcohol, parabens, urea derivatives, phenols, quaternary ammonia compounds, halogens, organomercury, or organic acids.

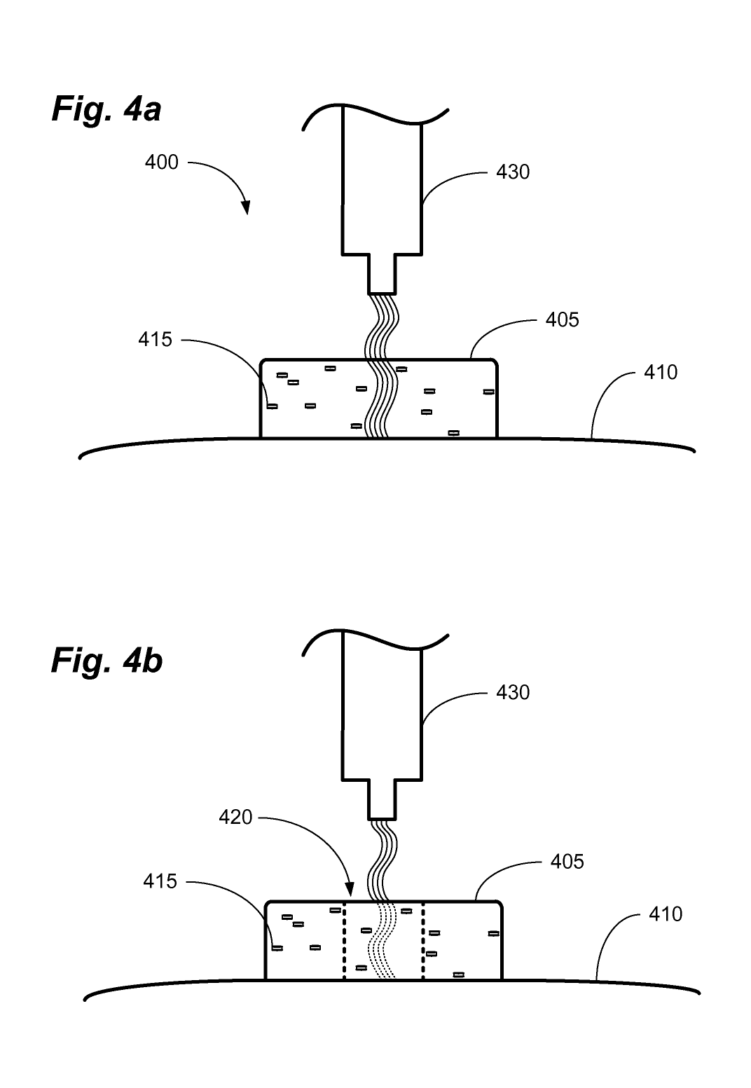

In some embodiments, as shown in FIG. 4a, the system 400 can comprise a patch 405 appliable to the treatment area 410 (e.g., the patient's skin). The patch 405 can comprise, for example, a hydrogel patch 405, a polymer support structure, nanofiber sheets, or other suitable material. The patch 405 can be placed on the patient's skin 410 using a suitable temporary adhesive or gel, for example, and can comprise a plurality of thermal capacitors 415.

In some embodiments, the patch 405 can be optically transparent, or transparent in a predetermined wavelength (i.e., the applicable wavelength or range of wavelengths of the treatment). In this manner the energy from the energy source 430 can penetrate the patch 405 substantially unimpeded. This can facilitate treatments that, for example, benefit from the light itself from the source 430, as opposed to light and heat, for effective treatment. In other words, the light from the source 430 can penetrate the patch 405, but the temperature of the entire surface 410 covered by the patch 405 can be controlled.

In other embodiments, shown in FIG. 4b, the patch 405 can be substantially toroidal and can include a hole 420. In this configuration, the patch 405 can be transparent, as before, or can be opaque in the relevant wavelength. In either configuration targeted treatment of a specific area on the patient's skin 410 can be provided, for example, while the remainder of the area is protected from the source 430 by the patch 405. In this manner, localized heating, when effective, can be applied, but collateral damage to surrounding tissue 410 that should not be treated is reduced or eliminated by the patch 405.

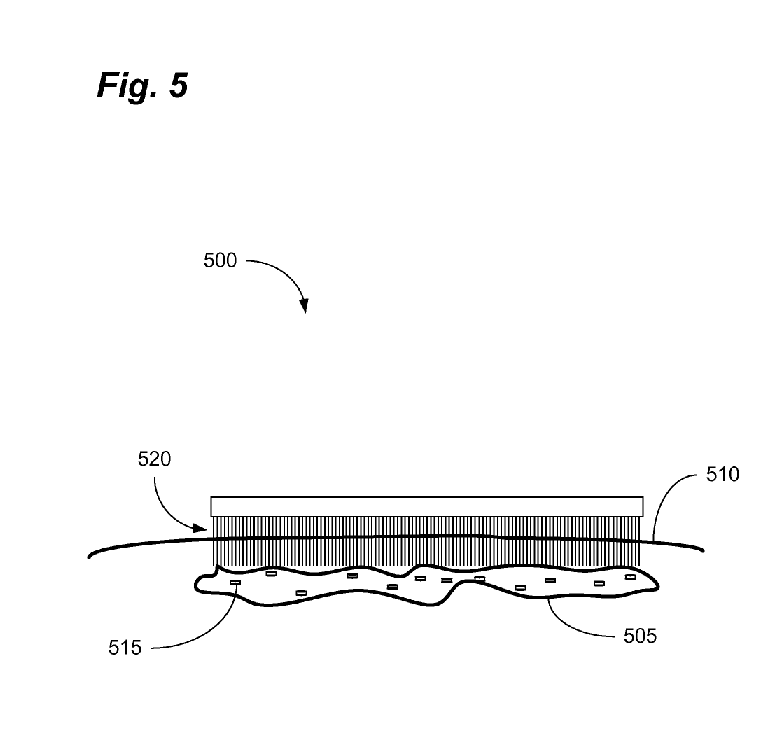

In still other embodiments, as shown in FIG. 5, the system 500 can comprise a plurality of thermal capacitors 515 suspended or dissolved in a liquid carrier 505. In this configuration, the carrier 505 can be, for example and not limitation, a liquid, cream, or gel with a sufficiently low viscosity such as water, glycerin, or glycerol, to enable it to be injected under the skin 510. In this manner, the system 500 can be injected under the skin using microneedle 520 injections, for example, to cool the skin 510 (or other tissue) from below.

This configuration 500 can be useful, for example, for treatments that penetrate the epidermis and reach the dermis or hypodermis, for example, for which topical application would be ineffective (or less effective). In this manner, the tissue 510 being treated can be cooled directly and the system 500 can be injected at the appropriate depth for the treatment. In this configuration, the system 500 can comprise components 505, 515 that are biocompatible and can be easily absorbed by the patient's body. In this configuration, the carrier 505 can be, for example and not limitation, water or saline; and, the thermal capacitors 515 can include, but are not limited to, salts, blood plasma, blood proteins, fats, and fatty acids.

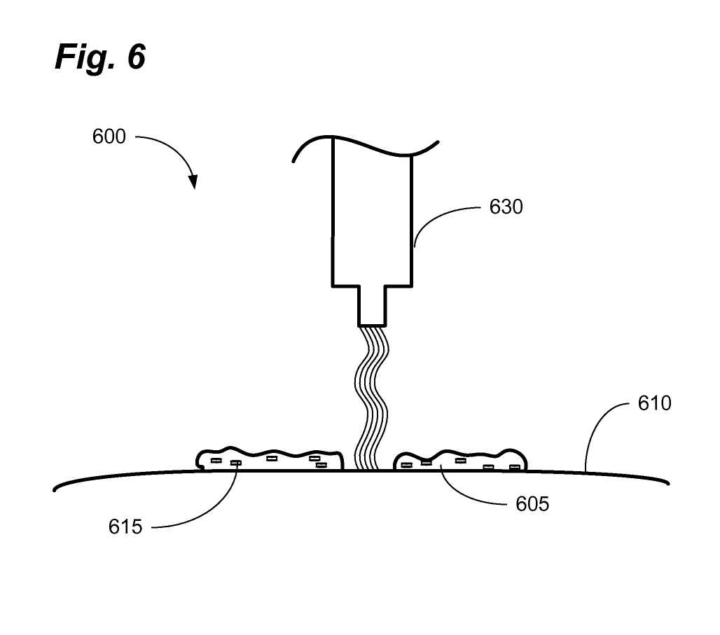

In still other embodiments, shown in FIG. 6, the system 600 can comprise a gel or cream 605, for example, to enable the system 600 to be applied only in areas of the tissue 610 that are not to be treated. In this manner, a suitably viscous gel or cream 605 containing a plurality of thermal capacitors 615 can be applied to areas of the skin 610, for example, that are not being treated enabling direct impingement of the energy source 630 on the skin 610 from the device 635. This configuration can be useful, for example, where localized heating is beneficial. In this manner, the area 610 for treatment can be heated appropriately by the source 630, but the temperature of surrounding tissue can be controlled.

### Example 1

To evaluate the ability of the system 100 to maintain skin temperatures below pain or injury thresholds, a prototype sample of the gel was spread across the surface of a thin film platinum heater that had been deposited on a 190 μm Silicon substrate. To prepare the prototype gel, 297 mg of Methyl palmitate (4.95%) was added to a flask with 6 mL of deionized water and heated to 33° C. for 10 min, until all of the substance had melted. 55 mg of xanthan gum (0.925%) was added to the flask with the melted methyl palmitate. The temperature of the mixture was maintained and stirred by hand until an even consistency was observed, approximately 15 min. The heater was then pulsed for 30 milliseconds once every 5 seconds. This pulse rate is representative of the periodic heating that would be produced by a laser, IPL device, or other light based medical device that provides transient heating.

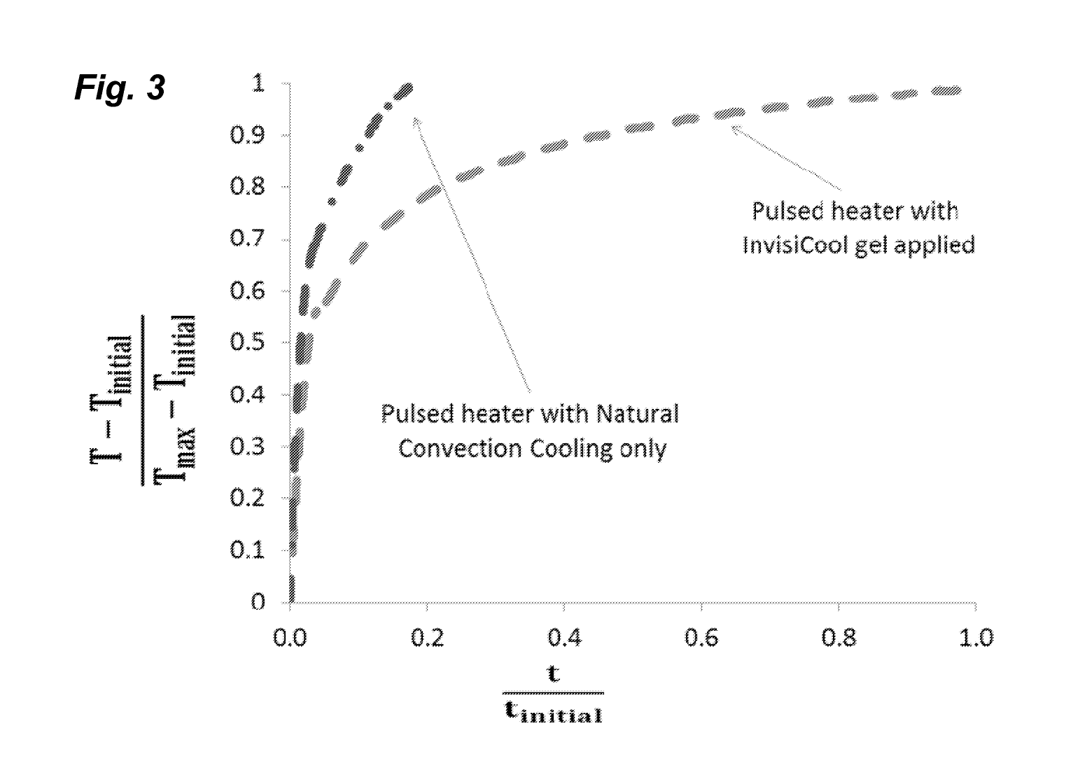

As shown in FIG. 3, at a heat flux of approximately 3.5 W/mm², the heater stayed below the threshold temperature for 5× longer than when the prototype gel was applied compared to simple convective heat dissipation. Obviously, increasing the amount of time the target site stays below the threshold temperature results in a decrease in cumulative pain and/or other side effects (e.g., scarring or swelling) experienced, as described above and shown schematically in FIG. 2. In addition, depending on the treatment being provided, longer or more intense treatments can result in improved, or faster, results for the patient.

Theoretical analysis using the first principle physical model of the process has also been conducted that shows that when high thermal conductivity materials are added to a PCM composite, the perimeter of the melted region is 1-2 diameters larger than the size of the heater when the threshold temperature is reached. This translates to an ability to keep the skin surrounding the treatment site cool, reducing any unnecessary collateral damage from the treatment.

A concern regarding the system 100 during medical procedures using a laser, or other light source, is the potential for the gel 100 to absorb or scatter the incident laser light. As a result, in some embodiments, it is advantageous to design the gel 100 to be minimally absorbing, particularly in the wavelength of the laser's irradiation. Unfortunately, while lasers emit substantially coherent light of a single wavelength, modern IPL systems typically emit over a much wider range (e.g., from approximately 500 nm to 1200 nm).

Fortunately, the main component of many gels and creams, including a hydrogel matrix, is water, which is very weakly absorbing in the visible and near infrared region and has an extinction path length of 1 cm or more below 1200 nm.⁴ Because the system 100 will generally be applied in sub-millimeter thin films, therefore, the impact of a water based matrix on light absorption can be negligible. In addition, from Beers law it can be determined that a PCM 110 loading below about 10% meets the acceptance criterion for light absorption regardless of their absorption characteristics. In some examples, the PCMs can also be made of a mixture of dielectric materials. In this configuration, their absorptivity can be made significantly less than 1, enabling significantly larger PCM 110 loading. This can enable the final composition to have a desirable combination of low laser absorption and high thermal storage. ⁴J. A. Curcio and C. C. Petty, “The near infrared absorption spectrum of liquid water,” JOSA, vol. 41, pp. 302-302, 1951.

While several possible embodiments are disclosed above, embodiments of the present invention are not so limited. For instance, while several possible configurations of materials for the carrier and the PCMs have been disclosed, other suitable materials and combinations of materials could be selected without departing from the spirit of embodiments of the invention. In addition, the various additives and components of embodiments of the present invention can be varied according to a particular application that requires a slight variation due to, for example, the type of procedure, the wavelength or intensity of the light used, or various hypoallergenic concerns. Such changes are intended to be embraced within the scope of the invention.

The specific configurations, choice of materials, and the size and shape of various elements can be varied according to particular design specifications or constraints requiring a device, system, or method constructed according to the principles of the invention. Such changes are intended to be embraced within the scope of the invention. The presently disclosed embodiments, therefore, are considered in all respects to be illustrative and not restrictive. The scope of the invention is indicated by the appended claims, rather than the foregoing description, and all changes that come within the meaning and range of equivalents thereof are intended to be embraced therein.

What is claimed is:

1. A system comprising:
a conformable matrix, with high thermal conductivity and high sensible heat capacity, appliable to a surface of a patient's body; and
a plurality of thermal capacitors with high latent heat capacity disposed in the conformable matrix such that there is high contact area between the thermal capacitors and the matrix;
wherein the thermal capacitors undergo an endothermic physico-chemical transformation above a first predetermined temperature and below a second predetermined temperature to control the temperature of a portion of the patient's body during a medical procedure.

2. The system of claim 1, wherein the thermal capacitors are phase change materials (PCMs); and
wherein the endothermic physico-chemical transformation is a phase change from a first state to a second state.

3. The system of claim 2, wherein the PCMs are solid and suspended in the conformable matrix below the first predetermined temperature; and
wherein the endothermic physico-chemical transformation is a phase change from solid to liquid above the first predetermined temperature.

4. The system of claim 2, wherein the PCMs are liquid and dissolved, dispersed, or both in the conformable matrix below the first predetermined temperature; and
wherein the endothermic physico-chemical transformation is a phase change from liquid to gas above the first predetermined temperature.

5. The system of claim 1, wherein the medical procedure is a light-based medical procedure in a first predetermined wavelength range; and
wherein the system is substantially transparent in the first predetermined wavelength range.

6. The system of claim 5, wherein the medical procedure is intense pulsed light (IPL) treatment; and
the first predetermined wavelength range is between approximately 500 nm and 1200 nm.

7. The system of claim 1, wherein the medical procedure is a laser treatment at a first wavelength; and
the first predetermined wavelength range is equal to the first wavelength.

8. The system of claim 1, wherein the system comprises materials that are one or more of hypoallergenic, non-toxic, or biocompatible.

9. The system of claim 2, wherein the PCMs comprise one or more selected from the group consisting of fatty acids, fatty acid esters, salt hydrates, and waxes.

10. The system of claim 1, further comprising one or more thickeners to adjust the rheological properties of the system.

11. The system of claim 1, further comprising one or more high thermal conductivity additives to enhance the thermal conductivity of the system.

12. A system comprising:
a patch, with high thermal conductivity and high sensible heat capacity, appliable to a surface of a patient's body;
a plurality of thermal capacitors with high latent heat capacity disposed in the patch such that there is high contact area between the thermal capacitors and the patch;
wherein the thermal capacitors undergo an endothermic physico-chemical transformation above a first predetermined temperature and below a second predetermined temperature to control the temperature of a portion of the patient's body during a medical procedure.

13. The system of claim 12, wherein the medical procedure is a light-based medical procedure in a first predetermined wavelength range; and
wherein the system is substantially transparent in the first predetermined wavelength range.

14. The system of claim 12, the patch further defining a hole disposed approximately in the center of the patch.

15. The system of claim 12, wherein the first predetermined temperature is approximately 20° C.

16. The system of claim 12, wherein the second predetermined temperature is between approximately 44-60° C.

17. A method comprising:
applying a gel to a portion of the patient's skin comprising:
a conformable, high thermal conductivity matrix, with high thermal conductivity and high sensible heat capacity; and
a plurality of thermal capacitors with high latent heat capacity disposed in the conformable matrix such that there is high contact area between the thermal capacitors and the matrix; and
exposing the patient's skin to a light-based medical treatment;
wherein the thermal capacitors undergo an endothermic physico-chemical transformation above a first predetermined temperature and below a second predetermined temperature to control the temperature of a portion of the patient's body during a medical procedure.

18. The method of claim 17, wherein the gel is only applied to the portion of the patient's skin that is not being treated with the light-based therapy.

19. The method of claim 17, wherein the light-based therapy comprises light in a first pre-determined wavelength range; and
the gel is substantially transparent in the first pre-determined wavelength range.

20. The system of claim 17, wherein applying the gel to a portion of the patient's skin comprise injecting the gel sub-epidermally with a plurality of microneedles.
